(19) United States
(12) Patent Application Publication
(10) Pub. No.: US 2007/0155023 A1
(43) Pub. Date: Jul. 5, 2007

(54) Identification of proteins and other species with emissive chelating label

(75) Inventors: Stephen J. Lippard, Cambridge, MA (US); Christian R. Goldsmith, Needham, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 11/637,957

(22) Filed: Dec. 12, 2006

Related U.S. Application Data
(60) Provisional application No. 60/750,083, filed on Dec. 13, 2005.

(51) Int. Cl. G01N 33/543; C07F 15/00

(52) U.S. Cl. 436/518; 546/2; 549/206; 548/101

(57) ABSTRACT

The present invention relates to compositions and methods for the emissive, e.g. fluorescent labeling of an analyte or other species. Emissive compositions may be attached to an analyte (e.g, a protein, a cell, or other biomolecule), where light emission from the composition may be used to determine the analyte. Emissive compositions of the present invention exhibit little or no significant decrease in light emission in the presence of metal ions.

### RELATED APPLICATIONS

This application claims priority under 35 U.S.C. § 119(e) to co-pending U.S. Provisional Application Ser. No. 60/750,083, filed Dec. 13, 2005, the contents of which are incorporated herein by reference.

### FIELD OF THE INVENTION

The present invention relates to compositions and methods for the fluorescent labeling of analytes, such as cells.

### BACKGROUND OF THE INVENTION

Fluorescence-based biological assays have demonstrated tremendous value in allowing researchers to understand complex biological processes. For example, fluorescent protein tags have facilitated the study of protein localization and dynamics within living cells, allowing researchers to study cellular responses to various disease-causing agents, resulting in improved treatment alternatives. While numerous methods of fluorescent protein labeling exist, many methods may require complex genetic manipulation to incorporate either a fluorescent peptide or a target into the native sequence for attachment of an exogenous fluorophore. The steric bulk of these additions can often disrupt the structure and activity of the protein.

Fluorescent proteins (FPs) have been widely used as labels due to their strong optical properties. FPs also have excellent biological compatibility because they may be genetically encoded and expressed by a cell itself. However, the poor photostability of some FPs can cause difficulties in long-term monitoring of cellular events, where high sensitivity and high image resolution are often desired. Additionally, the large size of FPs can prevent a critical component of the labeled construct from folding properly, thereby impacting function and localization. Also, the stability of wild type versions of common FPs can be difficult to control, as they have a tendency to oligomerize. Furthermore, many FPs are sensitive to pH, which can limit their ability to effectively image acidic regions of, for example, a cell. Genetic modification of FPs can potentially overcome such limitations, but this process typically is labor-intensive and time-consuming.

In some cases, such difficulties can be mitigated by using fluorescent small molecules, or synthetic probes, as labels. Generally, synthetic probes are less sterically demanding than fluorescent proteins and can be attached to the target peptide with minimal disruption of native structure and function. The photophysical and acid-base properties of fluorescent small molecules can be tailored relatively easily to suit an application of interest. However, available methods used to attach the probes to biological targets are somewhat limited. Additionally, attaching such probes to biological targets can compromise the performance of probes.

Accordingly, improved methods for fluorescent signaling of entities are needed.

### SUMMARY OF THE INVENTION

The present invention relates to compositions for fluorescent labeling of an analyte, comprising a fluorescent signaling entity linked to a chelating agent, the chelating agent having the ability to coordinate a metal ion via less than all available coordination sites of the metal, whereby the metal can be coordinated by a polyamino acid tag linked to an analyte for immobilization of the fluorescent signaling entity relative to the analyte, wherein the fluorescent signaling entity has a partial negative charge when in a medium at a pH in the range of about 5.0 to about 9.0 and the chelating agent, when bound to the metal ion, has a partial negative charge when in a medium at a pH in the range of about 5.0 to about 9.0.

The present invention also relates to compositions for fluorescent labeling of an analyte, comprising a fluorescent signaling entity linked to a chelating agent, the chelating agent having the ability to coordinate a metal ion via less than all available coordination sites of the metal, whereby the metal can be coordinated by a polyamino acid tag linked to an analyte for immobilization of the fluorescent signaling entity relative to the analyte, wherein the fluorescent signaling entity emits a signal that does not decrease or decreases by less than 40% in the presence of a paramagnetic metal ion, as compared to a signal emitted by the signaling entity in the absence of the paramagnetic metal ion under otherwise essentially identical conditions.

Another aspect of the present invention provides methods for determining an analyte, comprising exposing, to a medium suspected of containing the analyte, a composition comprising a fluorescent signaling entity linked to a chelating agent; in the event that the analyte is present, allowing the chelating agent to coordinate a metal ion, and allowing the analyte to become immobilized, via coordinative linking to the metal ion, to the fluorescent signaling entity; and determining a fluorescent signal emitted by the signaling entity, thereby determining the analyte, wherein the fluorescent signal emitted by the signaling entity immobilized with respect to the analyte via coordinative linking to the metal ion is at least 60% as intense as a signal emitted by the signaling entity in the absence of the metal ion under otherwise essentially identical conditions.

The present invention also relates to compositions for fluorescent labeling of an analyte, comprising a compound having the structure, 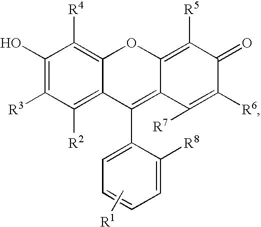
wherein R¹ comprises a chelating agent; R², R³, R⁴, R⁵, R⁶ and R⁷ can be the same or different and each is independently selected from among hydrogen, halide, cyano, sulfate, amino, hydroxyl, alkyl, heteroalkyl, alkenyl, heteroalkenyl, alkynyl, heteroalkynyl, aryl, heteroaryl, and substituted derivatives thereof; R⁸ is selected from among alkyl, heteroalkyl, alkenyl, heteroalkenyl, alkynyl, heteroalkynyl, carboxyl, sulfonate, and substituted derivatives thereof.

### BRIEF DESCRIPTION OF THE DRAWINGS

FIG. 1. shows (a) a fluorescein-nitrilotriacetic acid conjugate and (b) the synthesis thereof, according to one embodiment of the invention.

FIG. 2 shows the fluorescence emission spectra of a signaling entity (NTA-DCF) in the (a) absence of NiCl₂ and (b) presence of NiCl₂, according to one embodiment of the present invention.

FIG. 3 shows confocal microscopy images of HeLa cells labeled with (a) 5 μM NTA-DCF, (b) 5 μM NTA-DCF and 5 μM NiCl₂, and (c) 5 μM NiCl₂ upon exposure to ambient light (left images) and UV/visible light (right images).

FIG. 4 shows confocal microscopy images of HeLa cells transfected with Red Fluorescent Protein (mRFP) and an extracellular His₆ tagged protein. The emission from the NTA-DCF dye (left images) and from the mRFP (right images) for cells treated (a) 5 μM NTA-DCF and 5 μM NiCl₂ for 5 minutes, (b) 5 μM NTA-DCF for 5 minutes, (c) 5 μM NTA-DCF and 5 μM NiCl₂ in presence of EDTA for 5 minutes, and (d) 5 μM NTA-DCF and 5 μM NiCl₂ for 20 minutes are shown.

FIG. 5 shows confocal microscopy images of HEK 293-T cells transfected with Red Fluorescent Protein (mRFP) and either (a) pDisplay with extracellular His₆ tags and (bB) pDisplay without extracellular His₆ tags. The top images show NTA-DCF staining and the bottom images show both the emission from NTA-DCF and from mRFP.

FIG. 6 shows the negative-ion ESI spectrum of the NTA-DCF (LH₃) complex with Ni²⁺ in methanol.

FIG. 7 shows the nuclear morphology study of NTA-DCF toxicity, according to one embodiment of the invention.

### DETAILED DESCRIPTION

The present invention involves, in one aspect, the discovery and circumvention of a problem hindering certain categories of fluorescent labeling techniques and, in another aspect, the solution to that problem, involving compositions and methods.

In general, the invention relates to compositions and methods for the emissive (e.g., fluorescent) labeling of an analyte or other species (wherever “analyte” is used herein, it is to be understood that any other species that can be usefully attached to a fluorescent entity can take the place of the analyte). The invention involves techniques in which emissive compositions are attached or immobilized with respect to an analyte (e.g., a protein, a cell, biomolecule, or other molecule), where emission from the composition can be used to determine the analyte or otherwise provide a signaling or labeling effect. The compositions and methods described herein may be used in the emissive labeling of analytes, such as biomolecules, which may then be further used to assay biological or chemical entities, or combined with other detection techniques, in the study of the functions of biological systems. These general techniques are well known. In some cases, however, known fluorescent signaling entities lose effectiveness (fluorescence is diminished) in certain circumstances, particularly when brought into close proximity to certain metal ions, which may be inherent in the use of convenient linkage, as discussed below. Emissive compositions of the present invention, however, exhibit little or no decrease emission of electromagnetic radiation (e.g., visible light) in the presence of metal ions.

One reason that metal ions may inherently be brought into proximity of fluorescent signaling entities is that one class of convenient linkages for connecting fluorescent signaling entities with analytes or other species involves chelation of a metal ion by a multidentate ligand. Specifically, a fluorescent signaling entity may be linked (e.g., via a covalent bond) to a chelating agent, where the chelating agent has the ability to coordinate a metal ion via less than all available coordination sites of the metal, allowing the metal to further bind an analyte via, for example, a polyamino acid tag linked to the analyte. Via this linkage, the fluorescent signaling entity can become immobilized relative to the analyte.

Compositions of the present invention may be particularly advantageous due to their ability to substantially retain their emissive properties in the presence of, for example, paramagnetic metal ions. Fluorescent systems known in the art may generally exhibit a substantial decrease (e.g., 40% or greater) in fluorescence emission in the presence of a paramagnetic metal ion bound to the chelating agent relative to the fluorescence emission in the absence of the paramagnetic metal ion bound to the chelating agent, often due to quenching of the excited state fluorophore by the bound paramagnetic metal ion. As noted above, one aspect of the invention involves the discovery of a potential cause of the problem of quenching of fluorescent signaling entities by metal ions. Without wishing to be bound by any theory, the inventors point out that this quenching may be facilitated by attractive forces bringing the fluorophore and the paramagnetic metal ion into contact, or otherwise into close enough proximity with one another for quenching to occur at least to some extent. For example, a fluorescent signaling entity having a partial positive charge may be linked to a chelating agent having a partial negative charge when bound to the paramagnetic metal ion. The resulting attraction between these partial positive and partial negative charges may bring into contact or close proximity the chelating agent (hence, the paramagnetic metal ion) and the fluorescent signaling entity, resulting in the quenching of fluorescence emission. In one example, the use of paramagnetic ions, such as Ni²⁺, to bridge an analyte and a fluorescent tag have rendered the tags substantially non-fluorescent due to interaction with the paramagnetic metal ion. The decreased fluorescence emission may be problematic in cases where, for example, quantification of an analyte is desired, long-term monitoring of an analyte is required, or a low quantity of analyte is present.

As noted above, another aspect of the invention involves a solution to the quenching phenomenon described above, whereby emissive compositions of the present invention may be used in conjunction with metal ions (including paramagnetic metal ions) serving a role in linkage of the emissive composition to another species, without significant loss in effective optical properties (e.g., fluorescence emission). In one arrangement, this aspect of the invention involves emissive species that have at least a partial negative charge in their typical environments of use (described below). Again without wishing to be bound by theory, it is believed that these emissive compositions may repel partial negative charges carried by chelating agents, thereby keeping chelated metal ions distant enough to avoid or significantly inhibit their quenching by those ions, and thereby substantially avoiding loss of their emissive properties. In another arrangement, an emissive species is linked to a chelating agent via a linker of sufficient rigidity and length or other geometrical parameter (e.g., inherent steric properties), such that chelated metal ions are maintained at a sufficient distance from the emissive species to avoid or significantly inhibit quenching.

As used herein, a “partial negative charge” or “partial positive charge” on a molecule is given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way. A partial negative charge on a fluorescent signaling entity or other emissive species also can result if the species becomes fully or partially deprotonated in its environment of use. For example, under certain conditions (e.g. within an appropriate pH range that can be selected by one of ordinary skill in the art in conjunction with the signaling entity), a functional group on a fluorescent signaling entity may exist in equilibrium between a protonated state and a deprotonated state, wherein the equilibrium favors the deprotonated state and therefore a partial negative charge. In some embodiments, the partial negative charge similarly may be generated on a molecule comprising a bond between a hydrogen atom and an oxygen, nitrogen, or sulfur atom, or the like (e.g., hydroxyl, amine, thiol, sulfonate, etc.). For example, compositions of the present invention may comprise a phenol moiety and/or a carboxylic acid moiety on the emissive signaling entity, wherein at least one moiety may become deprotonated to generate a partial negative charge. A “partial negative charge,” as used herein, is a negative charge associated with a fluorescent signaling entity sufficient to repel a chelated metal atom, of the type described for use herein for linkage to a polyamino acid tagged entity, to the extent necessary to prevent or inhibit quenching of the fluorescent signaling entity by the metal atom. A “partial positive charge” can have the opposite effect.

Emissive signaling species of the invention can be characterized in exhibiting a first emission in the absence of a metal ion (e.g. a paramagnetic metal ion), and, in the presence of the metal ion, the emissive material may exhibit a second emission at least 60% as intense as the first emission.

As noted, emissive signaling entities in one set of embodiments have a partial negative charge in a typical environment of use. For example, they may have a partial negative charge in a medium at a pH in the range of about 5.0 to about 9.0, about 7.0 to about 8.0, or about 6.5 to about 7.5. Such signaling entities can be provided in conjunction with a linking chelating agent which, when bound to a metal ion, also has a partial negative charge in medium at a pH within a range as noted above. A partial negative charge associated with a chelating agent can be generated by, for example, deprotonation of at least one functional group of the chelating agent. Deprotonation of the chelating agent may occur upon chelation of the metal ion. In some cases, the chelating agent has a partial negative charge when exposed to a medium at a pH in the range of about 5.0 to about 9.0, about 7.0 to about 8.0, or about 6.5 to about 7.5. Examples of functional groups on the chelating agent which may generate a partial negative charge include carboxylic acids, alcohols, amines, thiols, sulfonamides, thioates (e.g., diphosphorothioates, and the like), phosphonates (e.g., alkylphosphonates, and the like), or amidates (e.g., phosphoroamidates).

Examples of chelating agents suitable for use in the invention which may generate a partial negative charge as described herein include, but are not limited to, nitrilotriacetic acid (NTA), iminodiacetic acid, macrocyclic amines (e.g., 1,4,7-triazacyclononane), crown ethers, polypyridyl groups, polyimidazolyl groups, other groups containing nitrogen heterocycles (e.g., purines), or combinations thereof. Any chelating agent suitable for linkage as described herein, regardless of the degree of partial negative charge carried, can be used in embodiments in which a linker that is rigid or of steric properties causing separation of chelating agent and signaling entity.

In one embodiment, an NTA moiety may bind to a metal ion such as Ni²⁺, wherein the carboxylic acid functional groups become deprotonated and coordinate to Ni²⁺, generating a partial negative charge on the NTA moiety. In one embodiment, a fluorescent signaling entity of the invention has a partial negative charge in medium at a pH of about 7.0, and the chelating agent, when bound to a metal ion, also has a partial negative charge in medium at a pH of about 7.0.

As noted, compositions of the present invention may emit substantially the same signal (or a signal reduced only slightly) in the presence of a paramagnetic metal ion as compared to their signal under conditions essentially identical but in the absence of the paramagnetic ion. The term “paramagnetic metal ion” is known in the art and refers to a metal ion having unpaired electrons, causing the metal ion to have a measurable magnetic moment in the presence of an externally applied field. Paramagnetic metal ions have been shown to substantially decrease or quench the signal emitted by a fluorescent signaling entity.

In some embodiments, the present invention comprises an emissive species such as a fluorescent signaling entity which, in the presence of a metal ion such as a paramagnetic metal ion, emits a signal that does not decrease or decreases by less than 40% as compared to the signal emitted in the absence of the paramagnetic metal ion under otherwise essentially identical conditions. In some cases, the signal decreases by less than 30%, less than 20%, less than 10%, or less than 5% in the presence of the metal ion under otherwise essentially identical conditions.

Another aspect of the invention provides methods for determining an analyte comprising exposing a composition containing a fluorescent signaling entity linked to a chelating agent to a medium suspected of containing the analyte. In the event that the analyte is present, the method involves allowing the chelating agent to coordinate a metal ion and allowing the analyte to become immobilized to the fluorescent signaling entity via coordinative linking to the metal ion. The fluorescent signal emitted by the signaling entity may then be determined, thereby determining the analyte. Methods and compositions disclosed herein may be tailored to suit a particular application, as selected by those skilled in the art. In some embodiments, methods of the present invention include the use of compositions which may permeate the cell membrane, for example, for binding target moieties within the cell. In some embodiments, methods of the present invention include the use of compositions which are impermeable to the cell membrane, for example, for selective labeling of proteins on the surface of the cell membrane.

In some embodiments, the use of compositions described herein allows the signal emitted by the signaling entity immobilized with respect to the analyte or other species, via coordinative linking to a metal ion, to be at least 60% as intense as a signal emitted by the signaling entity under essentially identical conditions but in the absence of the metal ion. In some embodiments, the signal emitted by the fluorescent signaling entity in the presence of the metal ion may be at least 70%, 80%, 90%, or 95% as intense as a signal emitted by the signaling entity in the absence of the metal ion.

The compositions and methods of the invention may be used with a broad range of fluorescent labeling systems that are not limited by, for example, the nature of the metal ion used in binding a fluorescent label to the analyte. The compositions may be tailored to be highly selective for an analyte, such as an analyte comprising or linked to a polyhistidine tag. Also, compositions of the invention utilized emissive small molecules which may less sterically demanding than other known labeling systems and can be attached to the target molecule with minimal disruption of native structure and function. Additionally, the photophysical and acid-base properties of the compositions can be relatively easily tailored to suit an application of interest.

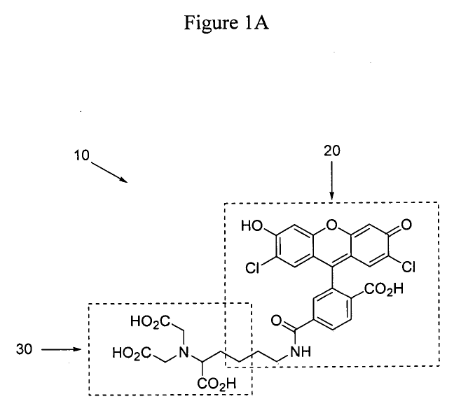

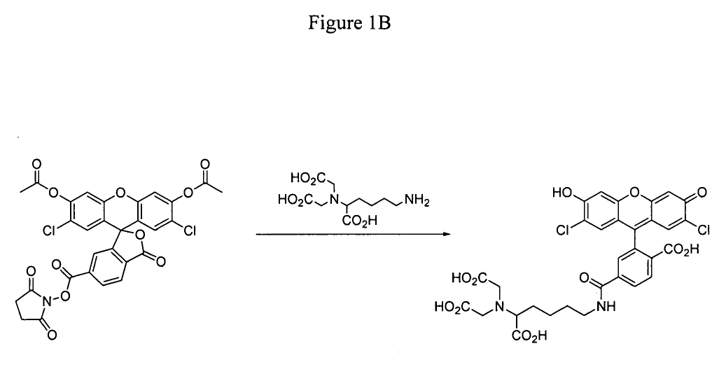

In an illustrative embodiment, FIG. 1A shows a composition according to one embodiment of the invention. Composition 10, herein referred to as NTA-DCF, comprises a fluorescent signaling entity 20 linked to chelating agent 30 via a covalent bond, and an alkyl chain separates the fluorescent signaling entity 20 from the chelating agent 30. Fluorescent signaling entity 20 comprises a fluorescein moiety having bright fluorescence emission, and chelating agent 30 comprises a nitrilotriacetic acid moiety (NTA), which may strongly bind metal ions, such as Ni²⁺, for example.

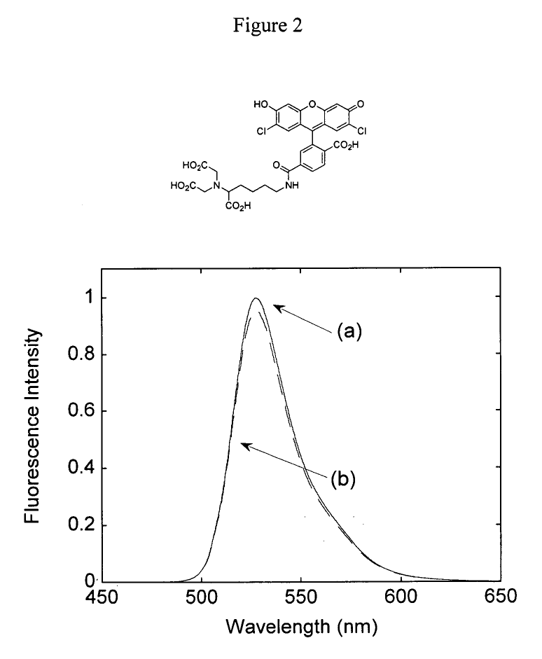

In an illustrative embodiment shown in FIG. 2, a fluorescent signaling entity, having a partial negative charge in a medium at a pH of about 7.0, may be covalently linked to a chelating agent that, when bound to a metal ion, also has a partial negative charge at a pH of about 7.0. The fluorescence emission spectrum of the signaling entity in the absence of metal ion is shown in FIG. 2A. FIG. 2B shows about a 5% decrease in fluorescence emission intensity upon the addition of NiCl₂. Consistent with the theory noted above, the chelating agent (bound to Ni²⁺) and the fluorescent signaling entity have partial negative charges and, as a result, repel each other, inhibiting quenching interaction between the fluorescent signaling entity and the paramagnetic metal ion. As a result, the fluorescent signaling entity emits substantially the same signal in the presence of a paramagnetic metal ion when compared to the signal emitted in the absence of a metal ion.

As noted, in one aspect, the invention provides a series of compositions comprising emissive entities. Examples of fluorescent entities suitable for use in the present invention include, but are not limited to, fluorescein, coumarin, rhodamine, anthracene, pyrene, dansyl, seminaphthofluorescein, seminaphthorhodamine, naphthofluorescein, naphthorhodamine, acridine, cyanine, BODIPY® (available from Invitrogen), Alexa Fluor® (available from Invitrogen), Cy3, Cy5.5, Cy7, and the like (available from Amersham Biosciences), Oregon Green, Pennsylvania Green, Tokyo Green, other fluorescent dyes, aryls, heteroaryls, derivatives thereof, and the like. Substituted derivatives or analogs of the above or other fluorescent signaling entities also can be used, as would be understood by those of ordinary skill in the art. Those of ordinary skill in the art will be able to select, from this list and from other suitable signaling entities, those that have a partial negative charge when in a medium at a pH as described herein, or those that can be readily modified so as to have such a partial negative charge. Such modification can include addition of functional groups such as carboxylic acids, hydroxyl groups, thiols, or the like. It is to be understood that fluorescent signaling entities can be used in conjunction with linkers having sufficient rigidity or steric properties that establish and maintain a sufficient distance between the fluorescent signaling entity and chelating agent to significantly inhibit or prevent quenching, in which case, the fluorescent signaling entity need not carry a partial negative charge.

In some embodiments, the present invention comprises compositions for fluorescent labeling of an analyte, comprising a general structure, 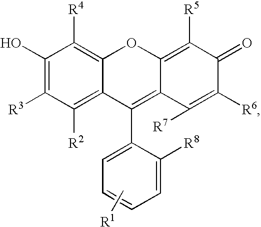
wherein R¹ comprises a chelating agent; R², R³, R⁴, R⁵, R⁶ and R⁷ can be the same or different and each is independently selected from among hydrogen, halide, cyano, sulfate, amino, alkylamino, hydroxyl, alkyl, heteroalkyl, alkenyl, heteroalkenyl, alkynyl, heteroalkynyl, aryl, heteroaryl, or substituted derivatives thereof; R⁸ is selected from among be alkyl, heteroalkyl, alkenyl, heteroalkenyl, alkynyl, heteroalkynyl, carboxyl, or sulfonate. R¹-R⁸ (R²-R⁸, where R¹ is a chelating agent as described below) are selected such that the composition has a partial negative charge under typical conditions of use, such as in contact with media within pH ranges noted above. In such a situation, the composition emits a fluorescent signal that does not decrease or decreases by less than 40%, less than 30%, less than 20%, less than 10%, or less than 5% in the presence of a paramagnetic metal ion as compared to the signal emitted by the signaling entity in the absence of the paramagnetic metal ion.

The identity of the R¹-R⁸ substituent may affect the fluorescence properties of the composition, as known to one of skill in the art. A variety of mechanisms may explain the affect of the substituents on fluorescence, often by quenching, including, for example, photoinduced electron transfer (PET) and electronic energy transfer (EET). Substituents may be selected based on their electronic properties. For example, substituents having unpaired electrons at the atom directly attached to the aromatic ring, such as an amine or alkoxy substituent, may result in quenching of the fluorescence of the fluorescein. In some embodiments, substituents having loosely bound electron pairs (e.g., substituents having low electronegativity), such as a thiol or an iodine, may result in quenching of the fluorescence of the fluorescein. However, in other embodiments, substituents which are highly electron-withdrawing, such as nitro, may also cause quenching. Those skilled in the art may select substituents or combinations of substituents which may impart the desired fluorescence properties.

In some cases, R¹ may comprise a chelating agent covalently linked to the fluorescein moiety via an acyl, alkyl, heteroalkyl, alkenyl, heteroalkenyl, alkynyl, heteroalkynyl, aryl, or heteroaryl group, via substituted derivatives thereof, or via combinations thereof. In one embodiment, R¹ comprises a chain, such as an alkyl or heteroalkyl chain, with the chelating agent attached to the terminal end of the chain. In one set of embodiments, R¹ may comprise a chelating agent covalently linked to the fluorescein moiety via a rigid linker, such as an aryl, heteroaryl, alkene, heteroalkene, alkyne, or heteroalkyne group. For example, the linker may be a phenyl, biphenyl, or xylyl group, an acetylene, an alkene, combinations thereof, and the like. As noted above, rigid linkers between the fluorescent signal entity and the chelating agent can inhibit and/or prevent quenching of the fluorescent signaling entity by a metal chelated by the chelating agent. Where such rigid linkers are used, they can have lengths, or can effectively separate the chelating agent from the fluorescent signaling entity by lengths, at least as great as that equal to the length of the molecule:

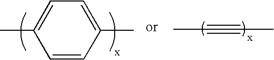

Where x is at least 2, or in other embodiments, 3, 4, 5, 6, 7, or greater. The structures above are not to be taken as limiting with respect to any type of linker that can be used, but simply as a comparative measure of the length of separation that the linker can provide, as measured on the molecular scale as would be understood by those of ordinary skill in the art. In this aspect of the invention, the linker can include one or more rigid portions and one or more non-rigid portions, so long as the combination of rigid and non-rigid portions of the linker separates the chelating agent from the fluorescent signaling entity by at least the distance noted above (as a comparative measure), even when non-rigid portions of the molecule allow the chelating agent and fluorescent signaling entity to come into closer proximity than the distance of the rigid portion itself. As used herein, a “rigid” portion means a portion of a molecule, the ends of which are separated by a distance which cannot change (outside of normal molecule-scale changes in temperature, etc.) without breaking at least one bond. For example, a portion of a molecule including sp³-hybridized carbon atoms will not be rigid (e.g., alkyl chains, and the like), while sp²-hybridized or sp-hybridized carbon atoms will impart a relatively higher degree of rigidity (e.g., aryl groups, alkynyl groups). Those of ordinary of skill in the art will understand this terminology.

In another set of embodiments the linker compels separation between the chelating agent and the fluorescent signaling entity by a distance as noted above via steric properties. For example, a portion of a molecule may allow for rotation about a carbon-carbon bond or the like which, in the absence of side groups and/or hetero atoms along the linker, might not allow the linker to provide the separation distance noted above, but, with sufficient of side groups and/or other chemical characteristics which those of ordinary skill in the art would be able to understand and select, define a “rigid” section providing the separation noted above. The chelating agent can be immobilized with respect to the overall composition in any of a variety of ways.

In some embodiments, R², R³, R⁴, R⁵, R⁶, and R⁷ may be a hydrogen, amino, alkylamino, halide, such as fluorine, chlorine, and bromine, hydroxyl, cyano, sulfate, alkyl, heteroalkyl, aryl, heteroaryl, or substituted derivatives thereof. In certain embodiments, R³ and R⁶ may be an electron-withdrawing group such as a halogen, such as chlorine.

In some embodiments, R⁴ and R⁵ may be hydrogen, amino, or alkylamino.

In some embodiments, R⁸ may be a carboxylic acid, a sulfonate, or an alkyl group, such as methyl.

In a particular embodiment, the composition comprises a compound having the structure, 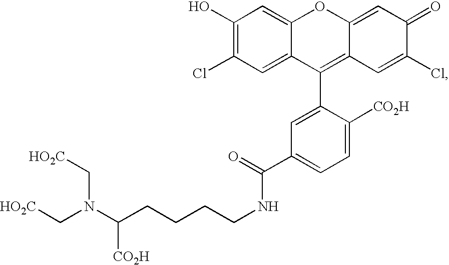
herein referred to as NTA-DCF. The NTA-DCF structure comprises a dichlorofluorescein moiety as the fluorescent signaling entity covalently linked to a nitrilotriacetic acid moiety as the chelating agent.

In general, compositions of the present invention may be synthesized using synthetic procedures known to those of ordinary skill in the art, or by straightforward modifications thereof, using readily available starting materials, reagents and conventional synthesis procedures. In an illustrative embodiment shown in FIG. 1B, 6-carboxy-2′,7′-dichlorofluorescein-3′,6′-diacetate-succinimidyl ester and N,N-bis(carboxymethyl)-L-lysine hydrate may form the product in a single step. The product may precipitate from acidified solution in high purity without need for further purification. Contemplated equivalents of the fluorescein-based compositions, as well as other compositions described herein, include such materials having the same general properties thereof, wherein one or more simple variations of substituents are made which do not adversely affect the efficacy of such molecule to achieve its intended purpose.

Although compositions of the invention find particular use in connection with immobilization with respect to other entities via coordination to a metal ion, use of the compositions is not limited in this way. Fluorescent compositions of the invention can serve their signaling or labeling function by being attached to an analyte or other species via a variety of linkages, such as a covalent bond (e.g. carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), or a dative bond (including but not limited to the above described complexation or chelation between metal ions and monodentate or multidentate ligands). In some cases, the emissive material and the analyte or other species may be linked via an interaction between a pair of two biological molecules.

Although specific embodiments of the invention are illustrated and described, those of ordinary skill in the art will recognize that the invention as claimed embraces a variety of different compositions which those of ordinary skill in the art would easily be able to design and construct based upon the teachings found herein.

Definitions and Other Explanatory Material

As used herein, a “signaling entity” refers to an entity that is capable of indicating its existence in a particular sample or at a particular location. Signaling entities of the invention can be those that are identifiable by the unaided human eye, those that may be invisible in isolation but may be detectable by the unaided human eye if in sufficient quantity, entities that absorb or emit electromagnetic radiation at a level or within a wavelength range such that they can be readily detected visibly (unaided or with a microscope including an electron microscope or the like), or the like. Examples include dyes, pigments, luminescent moieties (e.g., fluorescent, phosphorescent, or generally emissive moieties). A “fluorescent signaling entity” is a signaling entity which may be detectable via its fluorescence emission.

Upon exposure to electromagnetic radiation, the fluorescent signaling entity may have a fluorescence emission in the visible and/or UV range. In some embodiments, the fluorescent signaling entity may have a fluorescence emission from about 200 nm to about 800 nm, or from about 400 nm to about 700 nm.

A “polyamino acid tag” as used herein may refer to a type of affinity tag comprising a series of amino acids in proximity such that they can coordinate at least two free coordination sites of a metal ion. In some cases, a polyamino acid tag can become fastened to a metal that is coordinated by a chelating entity (e.g., NTA). In some embodiments, a polyamino acid tag may include neighboring amino acids such as, for example, neighboring histidines, lysines, arganines, glutamines, aspartines, or any combination thereof. The polyamino acid tag may include at least two, and, in some cases, from 2 to 10, or from 3 to 8, or 6, neighboring amino acids. When the polyamino acid tag includes histidine, it may be referred to as a “polyhistidine tag” or “histidine tag” or “HIS-tag,” and can be present at either the amino- or carboxy-terminus, or at any exposed region, of a peptide, protein, nucleic acid or other analyte or species. In one embodiment, the polyamino acid tag may be a six-residue poly-histidine (His₆), linked to an analyte. Those of ordinary skill in the art are aware of techniques for attaching polyamino acid tags to species using, e.g., traditional synthetic or genetic methods.

As described herein, the term “chelating agent” refers to an entity capable of coordinating a metal through two or more donor atoms. The chelating agent may be a molecule, such as an organic molecule, having two or more unshared electron pairs available for donation to a metal ion. The metal ion is usually coordinated by two or more electron pairs to the chelating agent. In some embodiments, the chelating agent may be a Lewis base. Chelating agents used in connection with the invention can be bidentate chelating agents, tridentate chelating agents, and/or tetradentate (or higher order) chelating agents, which refer to chelating agents having, respectively, two, three, and four (or more) electron pairs readily available for simultaneous donation to a metal ion coordinated by the chelating agent. Typically, the electron pairs of a chelating agent form coordinate bonds with a single metal ion. Those of ordinary skill in the art are capable of selecting an appropriate chelating agent and metal ion for the purpose of linking two molecules together, or linking a molecule or other species to a surface. In some cases, the chelating agent is selected to bind to a metal ion via less than all the available coordination sites of the metal ion. For example, a tridentate chelating agent may bind a metal ion having greater than three coordination sites.

In some embodiments, the chelating agent may be nitrilotriacetic acid, iminodiacetic acid, sulfonamide, diphosphorothioate, alkylphosphonate, or amidate. Nitrilotriacetic acid (NTA) binds a hexacoordinate metal ion via four coordination sites, leaving two available for linkage to a polyamino acid tag. Iminodiacetic acid can bind a monovalent metal cation, such as Cu(I). Other chelating agents known in the art may also be used, such that the chelate, when bound to a metal ion, retains a partial negative charge at a pH within a range noted above.

The metal ion that may bind compositions of the invention to an analyte via a chelating agent/metal ion/polyamino acid tag may be selected from those that have usually at least one, two, three, four, five, six, seven coordination sites or more. In some embodiments, the compositions and methods of the invention may be used with a wide range of metal ions, including light metals (Groups IA and IIA of the Periodic Table), transition metals (Groups IB-VIIIB of the Periodic Table), posttransition metals, metals of the lanthanide series and metals of the actinide series. In some embodiments, the metal ion is a paramagnetic metal ion. Examples of paramagnetic metal ions include, but are not limited to, Ni²⁺, Cr³⁺, Mn²⁺, Fe³⁺, Fe²⁺, Co²⁺, Cu²⁺, Pr³⁺, Nb³⁺, Sm³⁺, Yb³⁺, Eu³⁺, or Gd³⁺. It should be understood that the metal ions may have the appropriate spin-state and geometry to form paramagnetic metal ions (e.g., high-spin and octahedrally coordinated). In one embodiment, Ni²⁺ binds to a nitriloacetic acid chelating agent linked to a fluorescein moiety or other signaling entity, wherein the Ni²⁺ may further bind to a polyhistidine tag attached to an analyte when the analyte is present.

The term “determining” refers to quantitative or qualitative analysis of a species via, for example, spectroscopy, immunoassay, electrochemical measurement, and the like. “Determining” also means detecting or quantifying interaction between species, e.g. detection of binding between two species.

The term “sample” refers to any cell, tissue, or fluid from a biological source (a “biological sample”), or any other medium, biological or non-biological, that can advantageously be evaluated in accordance with the invention.

A “sample suspected of containing” a particular component means a sample with respect to which the content of the component is unknown.

The term “alkyl” is recognized in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” includes both “unsubstituted alkyls” and “substituted alkyls,” the latter of which refers to alkyl moieties having substituents replacing a hydrogen on one or more carbons of the hydrocarbon backbone. Such substituents may include, for example, a halogen, a hydroxyl, a carbonyl group (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. Those skilled in the art would recognize that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthiols, carbonyls (including ketones, aldehydes, carboxylates, and esters), CF₃, CN and the like. Cycloalkyls may be further substituted with alkyls, alkenyls, alkoxys, alkylthios, aminoalkyls, carbonyl-substituted alkyls, CF₃, CN and the like.

The term “heteroalkyl” is recognized in the art and refers to alkyl groups as described herein in which one or more atoms is a heteroatom (e.g., oxygen, nitrogen, sulfur, and the like).

The term “aliphatic” is recognized in the art and refers to linear, branched, and cyclic alkanes, alkenes, or alkynes. In certain embodiments, aliphatic groups in the present invention are linear or branched and have from 1 to about 20 carbon atoms.

The term “aralkyl” is recognized in the art and refers to alkyl groups substituted with an aryl group (e.g., an aromatic or heteroaromatic group).

The terms “alkenyl” and “alkynyl” are recognized in the art and refer to unsaturated aliphatic groups analogous in length and possible substitution to the alkyls described above, but that contain at least one double or triple bond respectively.

The “heteroalkenyl” and “heteroalkynyl” are recognized in the art and refer to alkenyl and alkynyl alkyl groups as described herein in which one or more atoms is a heteroatom (e.g., oxygen, nitrogen, sulfur, and the like).

The term “aryl” is recognized in the art and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl.” The aromatic ring may be substituted at one or more ring positions with such substituents as described herein. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocycles.

The terms “heterocycle” and “heterocyclic group” are recognized in the art and refer to 3- to about 10-membered ring structures, such as 3- to about 7-membered rings, whose ring structures include one to four heteroatoms. Heterocycles may also be polycycles. Examples of heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, CF₃, CN, or the like.

The terms “polycycle” and “polycyclic group” are recognized in the art and refer to structures with two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms, e.g., three or more atoms are common to both rings, are termed “bridged” rings. Each of the rings of the polycycle may be optionally substituted.

The term “nitro” is given its ordinary meaning in the art and refers to the group, NO₂.

The term “halogen” or “halide” is given its ordinary meaning in the art and refers to a fluorine, chlorine, bromine, or iodine.

The term “thiol” is given its ordinary meaning in the art and refers to the group, SH.

The term “hydroxyl” is given its ordinary meaning in the art and refers to the group, OH.

The term “cyano” is given its ordinary meaning in the art and refers to the group, CN.

The term “sulfate” is given its ordinary meaning in the art and refers to the group, SO₂.

The term “sulfonate” is given its ordinary meaning in the art and refers to the group, SO₃X, where X may be an electron pair, hydrogen, alkyl, cycloalkyl, or aryl.

The terms “amine” and “amino” are given their ordinary meaning in the art and refer to both unsubstituted and substituted amines, e.g., a moiety that may be represented by the general formulas NR′R″, wherein R′ and R″ each independently may be hydrogen, alkyl, heteroalkyl, alkenyl, heteroalkenyl, alkynyl, heteroalkynyl, aryl, heteroaryl, carbonyl, substituted derivatives thereof, or R′ and R″, taken together with the N atom to which they are attached complete a heterocycle or substituted heterocycle having from 4 to 8 atoms in the ring structure.

The term “alkylamino” is recognized in the art and refers to alkyl groups substituted with an amino group.

The term “carbonyl” is recognized in the art and refers to the group, C═O.

The terms “carboxyl,” “carboxyl group,” or “carbonyl group” is recognized in the art and can include such moieties as can be represented by the general formula: 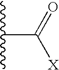
wherein X is H, OH, O-alkyl, O-alkenyl, or a pharmaceutically acceptable salt thereof. Where X is O-alkyl, the formula represents an “ester”. Where X is OH, the formula represents a “carboxylic acid”. In general, where the oxygen atom of the above formula is replaced by sulfur, the formula represents a “thiocarbonyl” group. Where X is a S-alkyl, the formula represents a “thiolester.” Where X is SH, the formula represents a “thiolcarboxylic acid.” On the other hand, where X is alkyl, the above formula represents a “ketone” group. Where X is hydrogen, the above formula represents an “aldehyde” group.

The term “substituted” is recognized in the art and includes all permissible substituents in accordance with permitted valence of the substituted atom and the substituent, and that the substitution results in a stable compound, e.g., which does not spontaneously undergo transformation such as by rearrangement, cyclization, elimination, or other reaction. In a broad aspect, the permissible substituents include acyclic and cyclic, branched and unbranched, carbocyclic and heterocyclic, aromatic and nonaromatic substituents of organic compounds. The permissible substituents may be one or more and the same or different for appropriate organic compounds. Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, CF₃, CN, or the like.

The terms “Lewis base” and “Lewis basic” are art-recognized and generally include a chemical moiety, a structural fragment or substituent capable of donating a pair of electrons under certain conditions. It may be possible to characterize a Lewis base as donating a single electron in certain complexes, depending on the identity of the Lewis base and the metal ion, but for most purposes, however, a Lewis base is best understood as a two electron donor. Examples of Lewis basic moieties include uncharged compounds such as alcohols, thiols, and amines, and charged moieties such as alkoxides, thiolates, carbanions, and a variety of other organic anions.

The terms “Lewis acid” and “Lewis acidic” are art-recognized and refer to chemical moieties which can accept a pair of electrons from a Lewis base as defined above.

The term “coordination” is recognized in the art and refers to an interaction in which one multi-electron pair donor coordinatively bonds (is “coordinated”) to one metal ion.

The terms “coordinate bond” or “coordination bond” are recognized in the art and refer to an interaction between an electron pair donor and a coordination site on a metal ion leading to an attractive force between the electron pair donor and the metal ion. The use of these terms is not intended to be limiting, in so much as certain coordinate bonds may also be classified as having more or less covalent character (if not entirely covalent character) depending on the nature of the metal ion and the electron pair donor.

The term “coordination site” is recognized in the art and refers to a point on a metal ion that can accept an electron pair donated, for example, by a solvent molecule or chelating agent.

The term “free coordination site” is recognized in the art and refers to a coordination site on a metal ion that is vacant or occupied by a species that is weakly donating. Such species is readily displaced by another species, such as a Lewis base.

The term “coordination geometry” is recognized in the art and refers to the manner in which coordination sites and free coordination sites are spatially arranged around a metal ion. Some examples of coordination geometry include octahedral, square planar, trigonal, trigonal biplanar and others known to those of skill in the art.

### EXAMPLES

The isolation and photophysical characterization of a probe (NTA-DCF) including a fluorescein reporter conjugated to an NTA-based polyhistidine targeting group is described herein. The compound is highly unusual in that it remains fluorescent (Φ=0.72) after chelation of a paramagnetic metal ion (e.g., Ni²⁺). This property allows NTA-DCF to act as a small yet highly fluorescent label for polyhistidine sequences in the presence of Ni²⁺. The probe is notable for its facile synthesis, the brightness of its emission, its small size relative to the commonly used FPs, and its ability to bind polyhistidine sequences selectively through its NTA targeting moiety.

General Procedure. Acetonitrile (MeCN) was dried over 3 Å molecular sieves. Triethylamine (Et₃N) and chloroform (CHCl₃) were purchased from Aldrich and used as received. 6-Carboxy-2′,7′-dichlorofluorescein-3′,6′-diacetatesuccinimidyl ester was synthesized by a method previously developed in this laboratory. ¹N,N-Bis(carboxymethyl)-L-lysine hydrate was purchased from Fluka and used as received. ¹H and ¹³C NMR spectra were acquired on either a Varian 300 Mhz or a Varian 500 MHz spectrometer and referenced to internal standards. IR spectra were collected on an Avatar 360 FT-IR instrument. Melting points were measured with a Mel-Temp apparatus. Fluorescence spectra were obtained on a Hitachi F-3020 spectrofluorimeter, and UV/vis spectra were acquired on a Cary 1E spectrophotometer. Low-resolution mass spectra were collected on an Agilent 1100 Series LC/MSD system. High-resolution mass spectra were provided by staff at the MIT Department of Chemistry Instrumentation Facility. Preliminary cell images were acquired with a Nikon Eclipse TS100 microscope equipped with an RT Diagnostics camera with illumination provided by a Chiu Mercury 100 W lamp. Magnification was 20×. The cell imaging system was operated with Spot Advanced software. Fluorescence images were obtained using a FITC-HYQ filter cube (excitation 460-500 nm, bandpass 510-560 nm). Select cell images were acquired with a LSM510 confocal microscope.

Aqueous solutions were prepared with Millipore water. A 2.25 mM stock solution of NTA-DCF in DMSO was prepared, separated into aliquots, stored at −25° C., and thawed in the dark immediately before use. All spectroscopic data were collected in a pH 7 buffer solution of 50 mM piperazine-N,N′-bis(2-ethanesulfonic acid) (PIPES) and 100 mM KCl unless noted otherwise. Solution magnetic susceptibilities were measured by the Evan's method. Spectroscopic data were taken at room temperature (22° C.). Quantum yields were measured relative to fluorescein in 0.1 M NaOH (Φ=0.95).

### Example 1

### Synthesis of N-[5-(Bis-carboxymethyl-amino)-5-carboxy-pentyl]-2-(2,7-dichloro-6-hydroxy-3-oxo-3H-xanthen-9-yl)-terephthalamic acid (NTA-DCF)

As shown in FIG. 1B, 6-Carboxy-2′,7′-dichlorofluorescein-3′,6′-diacetatesuccinimidyl ester (0.148 g, 236 μmol) was dissolved in 4 mL of MeCN. N,N-Bis(carboxymethyl)-L-lysine hydrate was dissolved in 4 mL of H₂O and 0.4 mL of Et₃N and was added dropwise to the solution of the ester. Upon stirring the reaction mixture at room temperature for 12 h, 1 N HCl was added until pH=1.0 was reached. The acidified solution was poured into 50 mL of CHCl₃ to precipitate the product, which was isolated as an orange powder (0.0538 g, 33%). Mp: 182° C. (dec). ¹H NMR (300 MHz, DMSO-d6): δ1.47 (6H, m), 3.18 (2H, m), 3.42 (5H, m) 6.74 (2H, s), 6.91 (2H, s), 7.67 (1H, s), 8.12 (2H, dd, J₁=32.7 Hz, J₂=8.7 Hz). ¹³C NMR (125 MHz, DMSO-d6): δ 8.642, 23.24, 28.64, 29.36, 53.61, 64.35, 93.96, 103.66, 109.99, 116.38, 119.41, 119.99, 122.23, 125.34, 128.45, 129.66, 141.15, 150.01, 155.27, 164.51, 167.76, 173.43, 174.12. R_{f} (Reverse Phase, MeOH)=0.00 as free ligand, 0.48 as Ni(II) complex. FT-IR (KBr, cm⁻¹): 3351 (m), 3064 (m), 2945 (m), 1744 (s), 1629 (s), 1502 (s), 1430 (s), 1340 (m), 1267 (s), 1221 (s), 1195 (m), 1089 (m), 1044 (w), 1021 (w), 959 (m), 875 (m), 798 (w), 752 (w), 697 (m). HRMS (ESI) Calcd MNa⁺, 711.0755; Found, 711.0737.

### Example 2

### Photophysical Properties of NTA-DCF

The photophysical properties of the signaling entity were measured to observe the effect that introduction of the NTA functionality may have on the absorbance and/or emission of the signaling entity.

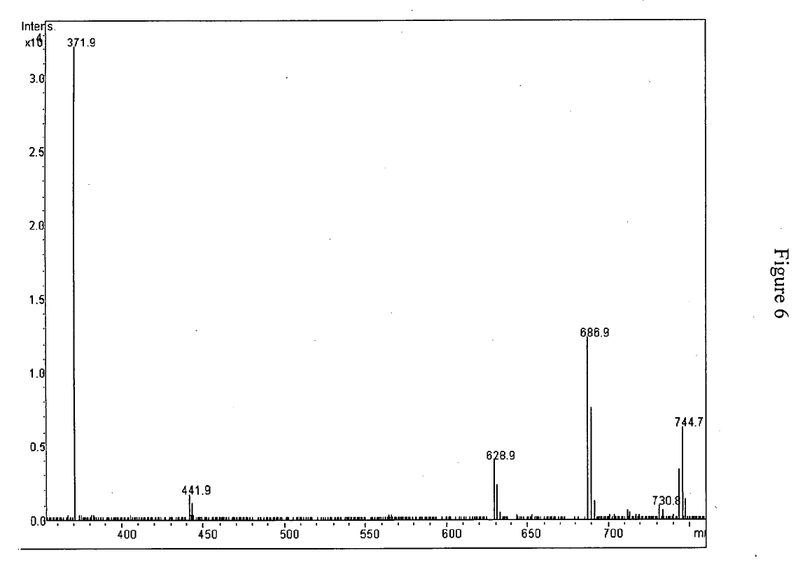

Neither the absorbance nor the emission of the fluorescein signaling entity were significantly impacted by the NTA functionality installed at the 6-position of the fluorescein ring. The absorption band for NTA-DCF at 504 nm (ε=86000 M⁻¹ cm⁻¹) and its emission at 527 nm lead to bright emission. The 504 nm absorption band for NTA-DCF was similar to that of 2′,7′-dichlorofluorescein. Similarly, the emission of NTA-DCF (λₘₐₓ=527 nm, Φ=0.78) resembled that of 2′,7′-dichlorofluorescein (λₘₐₓ=526 nm, Φ=0.88). Upon addition of NiCl₂ to a solution of NTA-DCF in 50 mM PIPES (piperazine-N,N′-bis(2-ethanesulfonic acid), 100 mM KCl, pH 7.0, the fluorescence decreases only minimally (5%, as shown in FIG. 2). Low-resolution mass spectrometry confirmed the complexation of Ni²⁺ to NTA-DCF. As shown in FIG. 6, the negative-ion ESI spectrum of the NTA-DCF (LH₃) complex with Ni²⁺ in methanol shows a peak at 744.7 m/z peak, which may correspond to the [(L)Ni(II)]¹⁻ species (calcd m/z=744.8). The 687.0 m/z peak may correspond to the [LH₂]¹⁻ species (calcd m/z=687.1). The 371.9 m/z peak may correspond to the [(L)Ni(I)]²⁻ species (calcd m/z=371.9). The solution magnetic susceptibility of 2.7(3) μ_{B} (292 K, MeOH-d4) was consistent with a d⁸ metal ion coordinated in an octahedral geometry, as anticipated from the known crystal structures of Ni²⁺ NTA complexes.

### Example 3

### Cell Culture and Transfection

This example describes the incorporation of an affinity tag within a target analyte (e.g., a cell). A polyhistidine affinity tag was incorporated within a plasmid, which was then used for transfection with the target cell. pDisplay-6×His was constructed by introduction of an oligonucleotide encoding for a His₆ tag (5′-CATCATCATCATCATCAT-3′) into a pDisplay plasmid (Invitrogen) digested with BglII and SalI.

Both HeLa and HEK 293-T cells were cultured in Dulbecco's Modified Eagle Medium (DMEM) supplemented with 10% fetal calf serum (FCS, Invitrogen), glutamine (2 mM), Penicillin (100 units/mL) and Streptomycin (100 μg/mL). The cells were passed and plated on 18 mm glass coverslips coated with 0.2% gelatin 36 hours before transfection. Both HeLa and HEK 293-T cells were transiently transfected with pDisplay (control plasmid) or the pDisplay-6×His (to install His₆ sequences on the extracellular side of the cell membrane) and pmRFP-C1 plasmids (for incorporation of Red Fluorescent Protein within the cell) in 2:1 ratio with use of Lipofectamine2000 (Invitrogen) according to manufacturer protocol.

### Example 4

### Labeling and Cell Permeability of Living Cells

Cells having affinity tags may then be fluorescently labeled with compositions of the present invention. The cells as described in Example 3 were used for labeling with NTA-DCF two days after transfection with plasmid. Immediately prior to labeling, the cells were washed once with PBS. A stock solution of complexed Ni²⁺-NTA-DCF was prepared by mixing 2.5 mM NiCl₂ and 2.5 mM NTA-DCF in a 1:1 ratio in PBS. HeLa and HEK 293-T cells were each washed once with PBS, then incubated with 5 μM Ni²⁺-NTA-DCF for 5 min at 22° C. in PBS. The cells were subsequently rinsed twice with PBS, fixed with 4% paraformaldehyde for 5 min, washed with two portions of PBS, and mounted using Vectashield anti-fading reagent (Vector Labs). As a negative control, a cell sample was incubated with NTA-DCF without added Ni²⁺. In order to gauge the reversibility of staining, 5 mM EDTA was added to a sample of cells stained with 5 μM NTA-DCF and 5 μM Ni²⁺. The NTA-DCF conjugate was observed to not enter either the HeLa or HEK 239-T cells, either alone or as its Ni²⁺ adduct. This impermeability can be exploited to label and image selectively proteins on the extracellular side of the cell membrane. The NTA-DCF composition was premixed with an equimolar concentration of NiCl₂, and the mixture was added to a sample containing either HeLa or HEK 293-T cells. NTA-DCF was shown to fluorescently label the polyhistidine sequences expressed on the extracellular plasma membrane surfaces of HeLa and HEK 293-T cells transfected with pDisplay plasmid containing a 6×His encoding sequence (pDisplay-6×His).

### Example 5

The fluorescently-labeled cells may be monitored by confocal microscopy. Confocal images of cells (NTA-DCF staining and mRFP fluorescence) were obtained with a Zeiss LSM510 microscope equipped with a 100× objective. Ar (488 nm) and HeNe (543 nm) lasers were used to provide the excitation for NTA-DCF and mRFP excitation, respectively. Each image was a z-series of 7-12 images taken at 0.75 μm depth intervals. Images of cells stained with Hoechst 33285 were obtained with a Zeiss Axioscope fluorescence microscope equipped with a 40× objective and a Hamamatsu digital camera.

### Example 6

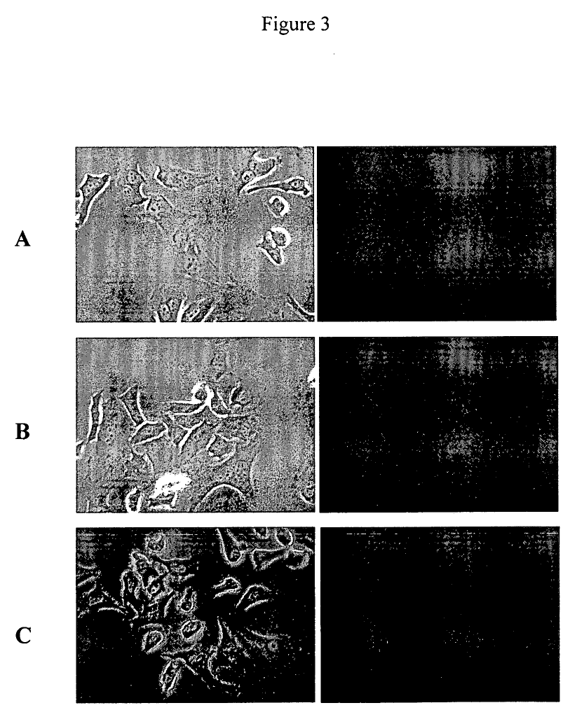

The fluorescently-labeled HeLa cells were observed using confocal microscopy. FIG. 3 shows the microscopy images of HeLa cells labeled with (A) 5 μM NTA-DCF, (B) 5 μM NTA-DCF and 5 μM NiCl₂, and (C) 5 μM NiCl₂ in regular light (left images) and fluorescent light (right images).

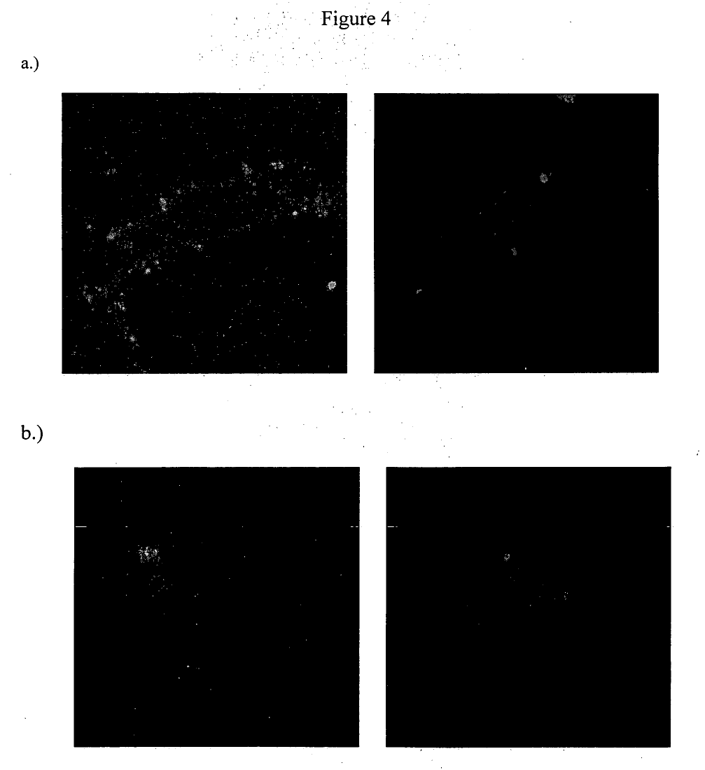

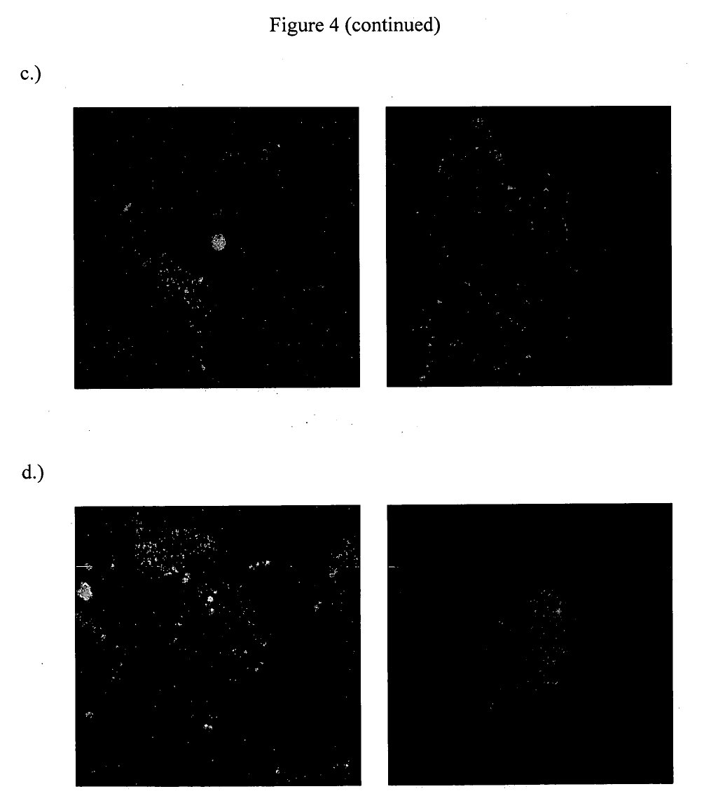

FIG. 4 shows images of HeLa cells transfected with Red Fluorescent Protein (mRFP) and extracellular His₆ tagged protein, immediately upon washing with PBS prior to fixation. The images on the left show the emission from added NTA-DCF dye. The images on the right show the emission from the mRFP. FIG. 4A shows cells which were treated with 5 μM NTA-DCF and 5 μM NiCl₂ for 5 min and then washed once with phosphate buffered saline (PBS). FIG. 4B shows cells which were treated with 5 μM NTA-DCF without NiCl₂ for 5 min. FIG. 4C shows cells which were treated with 5 μM NTA-DCF and 5 μM NiCl₂ for 5 min in presence of EDTA. FIG. 4D shows cells which were treated with 5 μM NTA-DCF and 5 μM NiCl₂ for 5 min. For FIG. 4D, the cells were fixed 20 minutes after washing with PBS, and the image was taken.

Observation of the confocal images shows that the HeLa cells do not express mRFP and pDisplay-6×His uniformly. In some cases, the cells only express mRFP, only pDisplay-6×His, or neither transfected sequence.

### Example 7

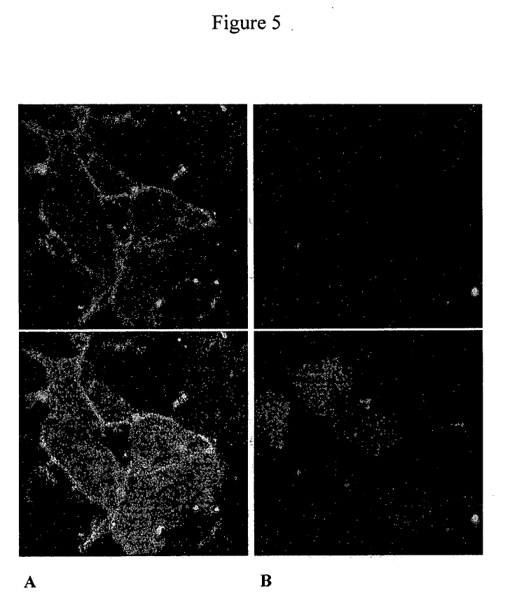

The fluorescently-labeled HEK 293-T cells were also observed using confocal microscopy. HEK 293-T cells were transfected with pDisplay-6His (extracellular His₆ tags), as shown in FIG. 5A, and pDisplay (without extracellular His₆ tags), as shown in FIG. 5B. As described herein, Red Fluorescent Protein (mRFP) was separately introduced to visualize the transfected cells. Upon 72 hr following transfection, the cells were stained for 5 min in phosphate buffered saline (PBS) containing 5 μM NTA-DCF and 5 μM NiCl₂ at 37° C. The cells were subsequently washed once with PBS and fixed with 4% PFA, 4% sucrose in PBS for 5 min, washed twice with PBS and mounted. The top images show the NTA-DCF staining while the bottom images covisualize the emission from NTA-DCF and mRFP.

Observation of the confocal images shows that the HEK 293-T cells do not express mRFP and pDisplay-6×His uniformly. In some cases, the cells only express mRFP, only pDisplay-6×His, or neither transfected sequence.

### Example 8

A metal chelator was added to observe the effect on binding of NTA-DCF probe to the target cell. The binding of the probe is blocked when Ni²⁺ is unavailable (FIG. 4B) or when the polyhistidine tag is not expressed (FIG. 5B), indicating that these two components are both required for fluorescent labeling. Consistent with these results, the addition of 5 mM of the extracellular metal chelator EDTA to the medium prevented Ni²⁺-NTA-DCF from binding to the cells (as in FIG. 4C, for example). In the absence of EDTA or a similar metal scavenging agent, the label-protein construct persisted for at least 20 min after washing, but its stability has not yet been fully assessed. It is likely that the stability is comparable to those of other His₆-Ni²⁺-NTA conjugates, for which the His₆-probe K_{d} value is in the 1-10 μM range.

### Example 9

### Nuclear Morphology Study of NTA-DCF Toxicity

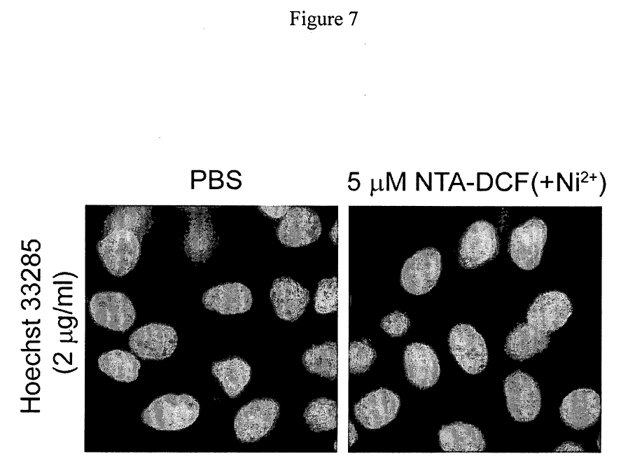

A nuclear morphology study suggests that the dye is not toxic at the 5-μM dose administered to the cell cultures (FIG. 7). Cells were incubated with either NTA-DCF or PBS for five min, washed twice with PBS, and fixed for 5 min with 4% p-formaldehyde. After fixation, the cells were rinsed four times with PBS (5 min each wash). Hoechst 33285 dye (Molecular Probes) was added to the second rinse such that the final concentration reached 2 μg/ml. Neither the size nor shapes of the nuclei change with the addition of NTA-DCF, suggesting that the cells are not dying in the presence of the dye.

Whereas several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, materials, reaction conditions, and configurations described herein are meant to be exemplary and that actual parameters, materials, reaction conditions, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood, unless otherwise indicated, to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently ““at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed:

1. A composition for fluorescent labeling of an analyte, comprising:
a fluorescent signaling entity linked to a chelating agent, the chelating agent having the ability to coordinate a metal ion via less than all available coordination sites of the metal, whereby the metal can be coordinated by a polyamino acid tag linked to an analyte for immobilization of the fluorescent signaling entity relative to the analyte,
wherein the fluorescent signaling entity has a partial negative charge when in a medium at a pH in the range of about 5.0 to about 9.0 and the chelating agent, when bound to the metal ion, has a partial negative charge when in a medium at a pH in the range of about 5.0 to about 9.0.

2. A composition as in claim 1, wherein the fluorescent signaling entity is covalently linked to the chelating agent.

3. A composition as in claim 1, wherein the fluorescent signaling entity is a fluorescent dye.

4. A composition as in claim 1, wherein the fluorescent dye comprises fluorescein, coumarin, rhodamine, acridine, cyanine, aryl, heteroaryl or a substituted derivative thereof.

5. A composition as in claim 1, wherein the fluorescent dye comprises fluorescein.

6. A composition as in claim 1, wherein the chelating agent comprises nitrilotriacetic acid, iminodiacetic acid, a sulfonamide, a diphosphorothioate, an alkylphosphonate, a macrocyclic amine, a crown ether, a heterocycle, an amidate, or a combination thereof.

7. A composition as in claim 1, wherein the chelating agent is nitrilotriacetic acid.

8. A composition as in claim 1, wherein the polyamino acid tag comprises histidine, lysine, arginine, glutamine, or combinations thereof.

9. A composition as in claim 1, wherein the polyamino acid tag is His₆.

10. A composition as in claim 1, wherein the analyte is a cell, protein, antibody, antigen, polymer, or ligand.

11. A composition as in claim 1, wherein the metal ion is Ni²⁺ or Zn²⁺.

12. A composition as in claim 1, wherein the metal ion is Ni²⁺.

13. A composition as in claim 1, wherein both the fluorescent signaling entity, and the chelating agent when bound to a metal ion, have a partial negative charge in a medium at a pH in a range of about 6.0 to about 8.0.

14. A composition as in claim 1, wherein both the fluorescent signaling entity, and the chelating agent when bound to a metal ion, have a partial negative charge in a medium at a pH in a range of about 6.5 to about 7.5.

15. A composition as in claim 1, wherein the fluorescent signaling entity emits a signal that does not decrease or decreases by less than 40% in the presence of a paramagnetic metal ion, as compared to a signal emitted by the signaling entity in the absence of the metal ion under otherwise essentially identical conditions.

16. A composition as in claim 1, wherein the fluorescent signaling entity emits a signal that does not decrease or decreases by less than 30% in the presence of a paramagnetic metal ion, as compared to a signal emitted by the signaling entity in the absence of the paramagnetic metal ion under otherwise essentially identical conditions.

17. A composition as in claim 1, wherein the fluorescent signaling entity emits a signal that does not decrease or decreases by less than 20% in the presence of a paramagnetic metal ion, as compared to a signal emitted by the signaling entity in the absence of the paramagnetic metal ion under otherwise essentially identical conditions.

18. A composition as in claim 1, wherein the fluorescent signaling entity emits a signal that does not decrease or decreases by less than 10% in the presence of a paramagnetic metal ion, as compared to a signal emitted by the signaling entity in the absence of the paramagnetic metal ion under otherwise essentially identical conditions.

19. A composition as in claim 1, wherein the fluorescent signaling entity emits a signal that does not decrease or decreases by less than 5% in the presence of a paramagnetic metal ion, as compared to a signal emitted by the signaling entity in the absence of the paramagnetic metal ion under otherwise essentially identical conditions.

20. A composition for fluorescent labeling of an analyte, comprising:
a fluorescent signaling entity linked to a chelating agent, the chelating agent having the ability to coordinate a metal ion via less than all available coordination sites of the metal, whereby the metal can be coordinated by a polyamino acid tag linked to an analyte for immobilization of the fluorescent signaling entity relative to the analyte,
wherein the fluorescent signaling entity emits a signal that does not decrease or decreases by less than 40% in the presence of a paramagnetic metal ion, as compared to a signal emitted by the signaling entity in the absence of the paramagnetic metal ion under otherwise essentially identical conditions.

21. A composition as in claim 20, wherein the fluorescent signaling entity is covalently linked to the chelating agent.

22. A composition as in claim 20, wherein the fluorescent signaling entity is a fluorescent dye.

23. A composition as in claim 20, wherein the fluorescent dye comprises fluorescein, coumarin, rhodamine, anthracene, pyrene, dansyl, seminaphthofluorescein, seminaphthorhodamine, naphthofluorescein, naphthorhodamine, acridine, cyanine, BODIPY®, Alexa Fluor®, Cy3, Cy5.5, Cy7, Oregon Green, Pennsylvania Green, Tokyo Green, aryl, heteroaryl, or a substituted derivative thereof.

24. A composition as in claim 20, wherein the fluorescent dye is fluorescein.

25. A composition as in claim 20, wherein the chelating agent comprises nitrilotriacetic acid, iminodiacetic acid, a sulfonamide, a diphosphorothioate, an alkylphosphonate, a macrocyclic amine, a crown ether, a heterocycle, an amidate, or a combination thereof.

26. A composition as in claim 20, wherein the chelating agent is nitrilotriacetic acid.

27. A composition as in claim 20, wherein the polyamino acid tag comprises histidine, lysine, arginine, glutamine, or combinations thereof.

28. A composition as in claim 20, wherein the polyamino acid tag is His₆.

29. A composition as in claim 20, wherein the analyte is a cell, protein, antibody, antigen, polymer, or ligand.

30. A composition as in claim 20, wherein the metal ion is Ni²⁺ or Zn²⁺.

31. A composition as in claim 20, wherein the metal ion is Ni²⁺.

32. A composition as in claim 20, wherein the fluorescent signaling entity emits a signal that does not decrease or decreases by less than 30% in the presence of a paramagnetic metal ion, as compared to a signal emitted by the signaling entity in the absence of the paramagnetic metal ion under otherwise essentially identical conditions.

33. A composition as in claim 20, wherein the fluorescent signaling entity emits a signal that does not decrease or decreases by less than 20% in the presence of a paramagnetic metal ion, as compared to a signal emitted by the signaling entity in the absence of the paramagnetic metal ion under otherwise essentially identical conditions.

34. A composition as in claim 20, wherein the fluorescent signaling entity emits a signal that does not decrease or decreases by less than 10% in the presence of a paramagnetic metal ion, as compared to a signal emitted by the signaling entity in the absence of the paramagnetic metal ion under otherwise essentially identical conditions.

35. A composition as in claim 20, wherein the fluorescent signaling entity emits a signal that does not decrease or decreases by less than 5% in the presence of a paramagnetic metal ion, as compared to a signal emitted by the signaling entity in the absence of the paramagnetic metal ion under otherwise essentially identical conditions.

36. A method for determining an analyte, comprising:
exposing, to a medium suspected of containing the analyte, a composition comprising a fluorescent signaling entity linked to a chelating agent;
in the event that the analyte is present, allowing the chelating agent to coordinate a metal ion, and allowing the analyte to become immobilized, via coordinative linking to the metal ion, to the fluorescent signaling entity; and
determining a fluorescent signal emitted by the signaling entity, thereby determining the analyte,
wherein the fluorescent signal emitted by the signaling entity immobilized with respect to the analyte via coordinative linking to the metal ion is at least 60% as intense as a signal emitted by the signaling entity in the absence of the metal ion under otherwise essentially identical conditions.

37. A method as in claim 36, wherein the fluorescent signaling entity is covalently linked to the chelating agent.

38. A method as in claim 36, wherein the fluorescent signaling entity is a fluorescent dye.

39. A method as in claim 36, wherein the fluorescent dye comprises fluorescein, coumarin, rhodamine, anthracene, pyrene, dansyl, seminaphthofluorescein, seminaphthorhodamine, naphthofluorescein, naphthorhodamine, acridine, cyanine, BODIPY®, Alexa Fluor®, Cy3, Cy5.5, Cy7, Oregon Green, Pennsylvania Green, Tokyo Green, aryl, heteroaryl, or a substituted derivative thereof.

40. A method as in claim 36, wherein the fluorescent dye comprises fluorescein.

41. A method as in claim 36, wherein the chelating agent comprises nitrilotriacetic acid, iminodiacetic acid, a sulfonamide, a diphosphorothioate, an alkylphosphonate, a macrocyclic amine, a crown ether, a heterocycle, an amidate, or a combination thereof.

42. A method as in claim 36, wherein the chelating agent is nitrilotriacetic acid.

43. A method as in claim 36, wherein the coordinative linking of the analyte to the metal ion occurs via a polyamino acid tag on the analyte.

44. A method as in claim 36, wherein the polyamino acid tag comprises histidine, lysine, arginine, glutamine, or combinations thereof.

45. A method as in claim 36, wherein the polyamino acid tag is His₆.

46. A method as in claim 36, wherein the analyte is a cell, protein, antibody, antigen, polymer, or ligand.

47. A method as in claim 36, wherein the metal ion is Ni²⁺ or Zn²⁺.

48. A method as in claim 36, wherein the metal ion is Ni²⁺.

49. A method as in claim 36, wherein the medium suspected of containing the analyte has a pH in the range of about 5.0 to about 9.0.

50. A method as in claim 36, wherein the medium suspected of containing the analyte has a pH in the range of about 6.0 to about 8.0.

51. A method as in claim 36, wherein the medium suspected of containing the analyte has a pH in the range of about 6.5 to about 7.5.

52. A method as in claim 36, wherein the fluorescent signal emitted by the signaling entity immobilized with respect to the analyte via coordinative linking to the metal ion is at least 70% as intense as a signal emitted by the signaling entity in the absence of the metal ion under otherwise essentially identical conditions.

53. A method as in claim 36, wherein the fluorescent signal emitted by the signaling entity immobilized with respect to the analyte via coordinative linking to the metal ion is at least 80% as intense as a signal emitted by the signaling entity in the absence of the metal ion under otherwise essentially identical conditions.

54. A method as in claim 36, wherein the fluorescent signal emitted by the signaling entity immobilized with respect to the analyte via coordinative linking to the metal ion is at least 90% as intense as a signal emitted by the signaling entity in the absence of the metal ion under otherwise essentially identical conditions.

55. A method as in claim 36, wherein the fluorescent signal emitted by the signaling entity immobilized with respect to the analyte via coordinative linking to the metal ion is at least 95% as intense as a signal emitted by the signaling entity in the absence of the metal ion under otherwise essentially identical conditions.

56. A composition for fluorescent labeling of an analyte, comprising:
a compound having the structure, 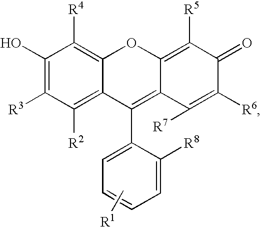
wherein R¹ comprises a chelating agent; R², R³, R⁴, R⁵, R⁶ and R can be the same or different and each is independently selected from among hydrogen, halide, cyano, sulfate, amino, hydroxyl, alkyl, heteroalkyl, alkenyl, heteroalkenyl, alkynyl, heteroalkynyl, aryl, heteroaryl, and substituted derivatives thereof; R⁸ is selected from among alkyl, heteroalkyl, alkenyl, heteroalkenyl, alkynyl, heteroalkynyl, carboxyl, sulfonate, and substituted derivatives thereof.

57. A composition as in claim 56, wherein R¹-R⁸ are selected such that the composition emits a fluorescent signal that does not decrease or decreases by less than 40% in the presence of a paramagnetic metal ion, as compared to a signal emitted by the signaling entity in the absence of the paramagnetic metal ion under otherwise essentially identical conditions.

58. A method as in claim 56, wherein the chelating agent is comprises nitrilotriacetic acid, iminodiacetic acid, a sulfonamide, a diphosphorothioate, an alkylphosphonate, a macrocyclic amine, a crown ether, a heterocycle, an amidate, or a combination thereof.

59. A method as in claim 56, wherein the chelating agent is nitrilotriacetic acid.

60. A composition as in claim 56, wherein the compound has the structure,

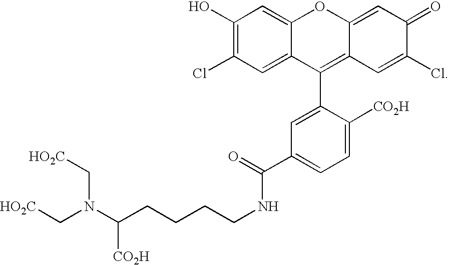

61. A composition for fluorescent labeling of an analyte, comprising:
a fluorescent signaling entity linked to a chelating agent via a linker, the chelating agent having the ability to coordinate a metal ion via less than all available coordination sites of the metal, whereby the metal can be coordinated by a polyamino acid tag linked to an analyte for immobilization of the fluorescent signaling entity relative to the analyte,
wherein the linker has sufficient length and rigidity to separate the fluorescent signaling entity from the chelating agent by a distance equal to at least: 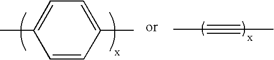
wherein x is at least 2.
